(19) United States
(12) Patent Application Publication
(10) Pub. No.: US 2018/0296551 A1
(43) Pub. Date: Oct. 18, 2018

(54) METHODS AND COMPOSITIONS FOR TREATING NEURODEGENERATIVE DISEASES

(71) Applicant: Ramot at Tel-Aviv University Ltd., Tel-Aviv (IL)

(72) Inventors: Beka SOLOMON, Herzlia Pituach (IL); Inna RABINOVICH-NIKITIN, Tel-Aviv (IL); Beka BARBIRO, Rishon-LeZion (IL); Assaf EZRA, Tel-Aviv (IL)

(21) Appl. No.: 15/769,095

(22) PCT Filed: Oct. 19, 2016
(86) PCT No.: PCT/IL2016/051133

§ 371 Date: Apr. 18, 2018

Related U.S. Application Data
(60) Provisional application No. 62/243,163, filed on Oct. 19, 2015.

(51) Int. Cl. A61K 31/495; A61K 9/00; A61P 25/28

(52) U.S. Cl. CPC A61K 31/495; A61K 9/0019; A61K 31/19; A61K 2300/00; A61P 25/28

(57) ABSTRACT

A method of treating a neurodegenerative disease is disclosed. The method comprises administering to the subject a therapeutically effective amount of a CXCR4 antagonist and lactate or/and zinc. Kits for treating same are also disclosed.

### FIELD AND BACKGROUND OF THE INVENTION

The present invention, in some embodiments thereof, relates to methods and compositions for treating neurodegenerative diseases and more particularly for treating Amyotrophic lateral sclerosis.

Amyotrophic lateral sclerosis (ALS) is the most common and most aggressive form of adult motor neuron (MN) degeneration. The pathophysiology of this neurodegenerative disease is complex which includes cholinergic deficit, glutamate excitatoxicity, neuroinflammation, immunity dysregulation, glucose hypometabolism and blood-central nervous system barrier (B-CNS-B) disruption.

Astrocytic cells are considered to play a primary role in the pathological process of amyotrophic lateral sclerosis (ALS), and are substantial contributors to motor neuron death. Astroglial abnormalities, such as changes in the release and uptake of astrocytic glutamate preface clinical symptoms of the disease (Vargas et al., 2010).

Chemokine receptors, including the G-protein-coupled receptor CXCR4, are expressed widely in neurons and glial cell. The ligand of CXCR4, the chemokine stromal-derived factor 1 (SDF-1), also known as CXCL12, evokes glutamate release and thereby modulates neuronal function or apoptosis. The mechanism of action includes increasing intracellular Ca2+ concentration, stimulation of extracellular signal related kinases and release of TNFα from astrocyte and microglia cell surface (Allen et al., 2001).

AMD3100 (1,1′-[4,4-Phenylenebis(methylene)]bis-1,4,8,11-tetraazacyclotetradecane) is a bicyclam molecule that specifically and reversibly blocks SDF-1 binding to CXCR4. AMD3100 has been shown to rapidly mobilize hematopoietic stem and progenitor cells (HSPCs) from the bone marrow (BM) into the blood of mice, non-human primates and humans. Disruption of CXCR4 signaling by AMD3100 was seen to inhibit the migration activity of grafted neuronal stem/progenitor cells, as observed in hemiplegic mice (Arimitsu et al., 2012). In 2008, AMD3100 was FDA-approved for HSPC mobilization in combination with granulocyte colony stimulating factor (G-CSF) in patients with non-Hodgkin's lymphoma and multiple myeloma undergoing autologous transplantation (Pusic et al., 2010).

Another substantial clinical feature of AMD3100 is the promotion of mobilization of CXCR4+VEGFR1+ cells through modulation of plasma SDF-1 levels, suggesting that AMD3100 plays a regulatory role in the recruitment of pro-angiogenic cells and in the extent of revascularization (Petit et al., 2007), which is important in maintenance and function of central nervous system (CNS) neurons.

In ALS patients and rodents expressing ALS-associated superoxide dismutase 1 (SOD1) mutations alterations were reported in the blood-Central Nervous System barrier (B-CNS-B) composed of the blood brain barrier (BBB), blood-spinal cord barrier (BSCB), and blood-cerebrospinal fluid barrier (BCSFB) as suggested from the reduction of levels of various tight junction proteins including ZO-1, occludin and claudin-5 between endothelial cells. The loss of tight junction proteins occludin and ZO-1 in the microvasculature has been also shown to be mediated by various cytokines such as monocyte chemoattractant protein-1 (MCP1), TNF-α, IL-1β, and IFN-γ. The reduction of the tight junction proteins resulted in microhemorrhage with release of neurotoxic hemoglobin-derived products, reductions in microcirculation and hypoperfusion. SOD1 mutants are proposed to mediate endothelial damage even before motor neuron death and hypoxia and inflammation led to increased BSCB permeability and disruption. Early motor-neuron dysfunction and injury were shown to be proportional to the degree of BSCB disruption, and early protection of the BSCB integrity was found to delay onset of motor-neuron impairment and degeneration. Altogether, these findings in mice show that BSCB breakdown plays a role in early-stage disease pathogenesis and that restoring BSCB integrity retards the disease process.

Additional background art includes PCT Application No. WO2015/031722 and Bridger et al., 1995, J Med Chem., 20;38(2):366-78.

### SUMMARY OF THE INVENTION

According to an aspect of some embodiments of the present invention there is provided a method of treating a neurodegenerative disease in a subject in need thereof comprising administering to the subject a therapeutically effective amount of a CXCR4 antagonist and lactate, thereby treating the neurodegenerative disease.

According to an aspect of some embodiments of the present invention there is provided a method of treating a neurodegenerative disease in a subject in need thereof comprising administering to the subject a therapeutically effective amount of AMD3100 and zinc, with the proviso that the neurodegenerative disease is not ALS, thereby treating the neurodegenerative disease.

According to an aspect of some embodiments of the present invention there is provided a kit for the treatment of a neurodegenerative disease comprising CXCR4 antagonist and lactate.

According to an aspect of some embodiments of the present invention there is provided a CXCR4 antagonist and lactate for use in treating a neurodegenerative disease.

According to an aspect of some embodiments of the present invention there is provided an AMD3100 and zinc for use in treating a neurodegenerative disease, with the proviso that the neurodegenerative disease is not ALS.

According to some embodiments of the invention, the neurodegenerative disease is selected from the group consisting of amyotrophic lateral sclerosis (ALS), Parkinson's disease (PD), Alzheimer's disease (AD), Huntington's disease, multiple sclerosis (MS), Creutzfeldt-Jacob disease (CJD), epilepsy, stroke, autoimmune encephalomyelitis, diabetic neuropathy and glaucomatous neuropathy.

According to some embodiments of the invention, the neurodegenerative disease is ALS.

According to some embodiments of the invention, the CXCR4 antagonist is selected from the group consisting of AMD3100 (plerixafor) BKT140, TN14003, CTCE-9908, KRH-2731, TC14012, KRH-3955, and AMD070.

According to some embodiments of the invention, the CXCR4 antagonist is AMD3100.

According to some embodiments of the invention, the lactate is administered concomitantly with the CXCR4 antagonist.

According to some embodiments of the invention, the lactate is administered prior to or following the CXCR4 antagonist.

According to some embodiments of the invention, the dose of the AMD3100 is less than 240 μg/kg.

According to some embodiments of the invention, the dose of the AMD3100 is between 0.1-500 μg/kg.

According to some embodiments of the invention, the dose of the AMD3100 is between 10-150 μg/kg.

According to some embodiments of the invention, the CXCR4 antagonist is administered subcutaneously.

According to some embodiments of the invention, the method further comprises administering to the subject zinc.

According to some embodiments of the invention, the AMD3100 is complexed with zinc.

According to some embodiments of the invention, the neurodegenerative disease is selected from the group consisting of Parkinson's disease (PD), Alzheimer's disease (AD), Huntington's disease, multiple sclerosis (MS), Creutzfeldt-Jacob disease (CJD), epilepsy, stroke, autoimmune encephalomyelitis, diabetic neuropathy and glaucomatous neuropathy.

According to some embodiments of the invention, the neurodegenerative disease is ALS.

According to some embodiments of the invention, the zinc is administered concomitantly with the AMD3100.

According to some embodiments of the invention, the zinc is complexed with the AMD3100 prior to the administering.

According to some embodiments of the invention, the zinc is administered prior to, or following the AMD3100.

According to some embodiments of the invention, the method further comprises administering to the subject lactate.

According to some embodiments of the invention, the dose of the AMD3100 is less than 240 μg/kg.

According to some embodiments of the invention, the dose of the AMD3100 is between 0.1-500 μg/kg.

According to some embodiments of the invention, the dose of the AMD3100 is between 10-150 μg/kg.

According to some embodiments of the invention, the AMD3100 is administered subcutaneously.

According to some embodiments of the invention, the CXCR4 antagonist is AMD3100.

According to some embodiments of the invention, the AMD3100 is complexed to zinc.

According to some embodiments of the invention, the kit further comprises zinc.

According to some embodiments of the invention, the neurodegenerative disease is selected from the group consisting of amyotrophic lateral sclerosis (ALS), Parkinson's disease (PD), Alzheimer's disease (AD), Huntington's disease, multiple sclerosis (MS), Creutzfeldt-Jacob disease (CJD), epilepsy, stroke, autoimmune encephalomyelitis, diabetic neuropathy and glaucomatous neuropathy.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a survival plot of 50 day old female SOD1-G93A mice following various treatments. PBS; 5 mg/kg AMD3100; 5 mg/kg AMD3100+896 mg/kg lactate, 896 mg/kg lactate. Median PBS treatment is 131 days; median 5 mg/kg AMD3100 treatment is 144 days; median 5 mg/kg AMD3100+896 mg/kg lactate treatment is 141 days; median 896 mg/kg lactate is 148 days. According to the survival plot of female mice it can be concluded that 50% of the mice treated with 5 mg/kg AMD3100+896 mg/kg lactate extend their life span to minimum 138 days old and maximum 149 days old. (P value <0.0001 performed by Mantel-Cox test). According to the probability of survival, 100% of mice treated with 5 mg/kg AMD3100+896 mg/kg lactate only start to die at day 138, whereas all PBS mice are dead by this age.

FIG. 2 is a graph illustrating weight change following the various treatments in 50 day old female SOD1-G93A mice. 5 mg/kg AMD3100; PBS; 5 mg/kg AMD3100+896 mg/kg lactate; 896 mg/kg lactate.

FIG. 3 is a graph illustrating change in motor function following the various treatments in 50 day old female SOD1-G93A mice. 5 mg/kg AMD3100; PBS; 5 mg/kg AMD3100+896 mg/kg lactate; 896 mg/kg lactate.

FIGS. 4A-B illustrate the change in MCT1 levels following 5 mg/kg AMD3100 treatment in 50 day old female SOD1-G93A mice. FIG. 4A shows increase in MCT1 levels in spinal cord. FIG. 4B shows MCT1 levels in muscles. The control group is set to 100%. Results are mean ±S.E.M, T-test; *p<0.05.

FIG. 5 illustrate the change in MCT1 levels following 5 mg/kg AMD3100 treatment versus 5 mg/kg AMD3100+896 mg/kg lactate treatment in 50 day old female littermate mice of SOD1-G93A mice. Littermate mice of SOD1-G93A treated with 5 mg/kg AMD3100 starting at 50 days old showed significant increase in MCT1 levels compared to non-treated mice.

FIGS. 6A-B illustrate the change in MBP and BACE1 following 5 mg/kg AMD3100 treatment versus 5 mg/kg AMD3100+896 mg/kg lactate treatment in 50 day old female mice of SOD1-G93A mice. FIG. 6A shows MBP levels. FIG. 6B shows BACE1 levels. Results are mean ±S.E.M. T-test; *p<0.05.

FIGS. 7A-B illustrate the change in activation of astrocytes following 5 mg/kg AMD3100 treatment versus 5 mg/kg AMD3100+896 mg/kg lactate treatment in 50 day old female mice of SOD1-G93A mice. FIG. 7A shows GFAP levels. FIG. 7B shows S100B levels. Results are mean ±S.E.M. T-test; *p<0.05.

FIGS. 8A-B illustrate the change in microglial reactivity following 5 mg/kg AMD3100 versus 5 mg/kg AMD3100+896 mg/kg lactate treatment in 50 day old female mice of SOD1-G93A mice. FIG. 8A shows Iba-1 levels. FIG. 8B shows IL-6 levels. Results are mean ±S.E.M. T-test; *p<0.05.

FIG. 9 is a survival plot of female SOD1-G93A mice treated with 0.25 mg/kg AMD3100+0.08 mg/kg Zn or 0.125 mg/kg AMD3100+0.04 mg/kg Zn at 50 days old. PBS; 5 mg/kg AMD3100; 0.25 mg/kg AMD3100+0.08 mg/kg Zn; 0.125 mg/kg AMD3100+0.04 mg/kg Zn. Median PBS treatment is 131 days; median 5 mg/kg AMD3100 treatment is 144 days; median 0.25 mg/kg AMD3100+0.08 mg/kg Zn treatment is 143 days; median 0.125 mg/kg AMD3100+0.04 mg/kg Zn treatment is 148 days. (P value <0.0001 performed by Mantel-Cox test). According to the probability of survival, 100% of mice treated with AMD3100-zinc only start dying after 141 days, whereas all PBS mice are dead by this age.

FIG. 10 is a graph illustrating weight change following the various treatments in 50 day old female SOD1-G93A mice. 5 mg/kg AMD3100; PBS; 0.25 mg/kg AMD3100+0.08 mg/kg Zinc.

FIG. 11 is a graph illustrating change in motor function following the various treatments in 50 day old female SOD1-G93A mice. PBS; 5 mg/kg AMD3100; 0.25 mg/kg AMD3100+0.08 mg/kg Zn.

FIG. 12 is a survival plot of 50 day old male SOD1-G93A mice following various treatments. PBS; 5 mg/kg AMD3100; 0.5 mg/kg AMD3100+0.17 mg/kg Zn+896 mg/kg lactate. Median PBS treatment is 126 days; median 5 mg/kg AMD3100 treatment is 140 days; median 0.5 mg/kg AMD3100+0.17 mg/kg Zn+896 mg/kg lactate treatment is 145 days. According to the survival plot, there is a 5 day extension of survival of 0.5 mg/kg AMD3100+0.17 mg/kg Zn+896 mg/kg lactate group, compared with 5 mg/kg AMD3100 treatment alone, and 19 days extension, compared with PBS (P value <0.0001 performed by Mantel-Cox test).

FIGS. 13A-B illustrate the change in inflammation following 5 mg/kg AMD3100 versus 0.25 mg/kg AMD3100+0.08 mg/kg zinc treatment in 50 day old female mice of SOD1-G93A mice. FIG. 13A shows GFAP levels. FIG. 13B shows Iba-1 levels. Results are mean ±S.E.M. T-test; *p<0.05

FIGS. 14A-B is a survival plot of 50 day old female and male SOD1-G93A mice following various treatments. PBS; 5 mg/kg AMD3100; 0.125 mg/kg AMD3100+0.04 mg/kg zinc+896 mg/kg lactate. FIG. 14A. Median female PBS treatment is 131 days; median female 5 mg/kg AMD3100 treatment is 144 days; median female 0.125 mg/kg AMD3100+0.04 mg/kg zinc+896 mg/kg lactate treatment is 144 days. FIG. 14B. Median male PBS treatment is 132 days; median male 5 mg/kg AMD3100 treatment is 140 days; median male 0.125 mg/kg AMD3100+0.04 mg/kg zinc+896 mg/kg lactate treatment is 151 days. (P value <0.0001 performed by Mantel-Cox test).

FIGS. 15A-B are bar graphs illustrating the Y maze results of female 3×Tg-AD mice treated with PBS; 0.5 mg/kg AMD3100 (AMD); 0.5 mg/kg AMD3100+0.17 mg/kg Zn (AMD+Zn); 0.5 mg/kg AMD3100+0.17 mg/kg Zn+896 mg/kg lactate (AMD+complex). A. Frequency of entries to the novel arm. B. Time spent in novel arm.

FIGS. 16A-B are bar graphs illustrating the changes in PHF-1 and MCT-1 results of 3×Tg-AD mice treated with PBS; 0.5 mg/kg AMD3100 (AMD); 0.5 mg/kg AMD3100+0.17 mg/kg Zn (AMD+Zn); 0.5 mg/kg AMD3100+0.17 mg/kg Zn+896 mg/kg lactate. A (AMD+complex). A. reduction in PHF-1 is pronounced in AMD+Zn and AMD+complex compared to AMD only B. A trend for increase in MCT-1 was observed, but was not statistically significant. Results are mean ±S.E.M. T-test; *p<0.05, **p<0.01, ***p<0.001.

FIGS. 17A-B are bar graphs illustrating the changes in myelin binding protein (MBP) isotypes. Results of 3×Tg-AD mice treated with PBS; 0.5 mg/kg AMD3100 (AMD); 0.5 mg/kg AMD3100+0.17 mg/kg Zn (AMD+Zn); 0.5 mg/kg AMD3100+0.17 mg/kg Zn+896 mg/kg lactate. A (AMD+complex). A. Increase in MBP-23 kDa isotype. B. Increase in MBP-18 kDa isotype. Results are mean ±S.E.M. T-test; *p<0.05, **p<0.01.

FIG. 18 are bar graphs illustrating the changes in ChAT. Results of 3×Tg-AD mice treated with PBS; 0.5 mg/kg AMD3100 (AMD); 0.5 mg/kg AMD3100+0.17 mg/kg Zn (AMD+Zn); 0.5 mg/kg AMD3100+0.17 mg/kg Zn+896 mg/kg lactate. Increase in ChAT was statistically significant in AMD+Zn and AMD+complex treatment groups only. Results are mean ±S.E.M. T-test; **p<0.01.

FIG. 19 are bar graphs illustrating the changes in AP by ELISA. Results of 3×Tg-AD mice treated with PBS; 0.5 mg/kg AMD3100 (AMD); 0.5 mg/kg AMD3100+0.17 mg/kg Zn (AMD+Zn); 0.5 mg/kg AMD3100+0.17 mg/kg Zn+896 mg/kg lactate. Reduction in AP was statistically significant in AMD and AMD+complex treatment groups in the membrane fraction. Results are mean ±S.E.M. T-test; **p<0.01.

### DESCRIPTION OF SPECIFIC EMBODIMENTS OF THE INVENTION

The present invention, in some embodiments thereof, relates to methods and compositions for treating neurodegenerative diseases and more particularly for treating Amyotrophic lateral sclerosis.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

CXCR4 is expressed by cells of immune system and the central nervous system. Signaling in response to CXCL12 binding triggers migration and recruitment of immune cells including T cells and monocytes to brain as well as migration of neurons and oligodendrocyte precursor cells.

It has previously been shown that an antagonist of CXCR4 (AMD3100) significantly increased the survival of SOD1-G93A transgenic mice (an animal model of amyotrophic lateral sclerosis (ALS)) and further delayed disease onset and improved their motor function. In addition, AMD3100 was shown to have beneficial effect on blood-spinal cord-barrier (BSCB) integrity restoration, increased remyelination markers and reduced inflammation.

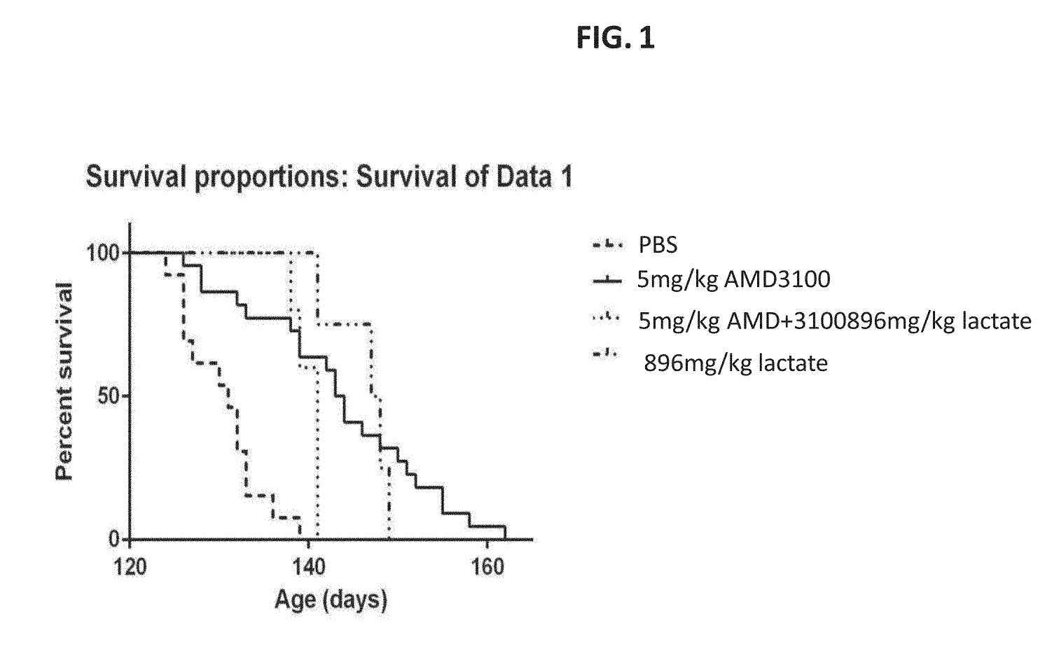

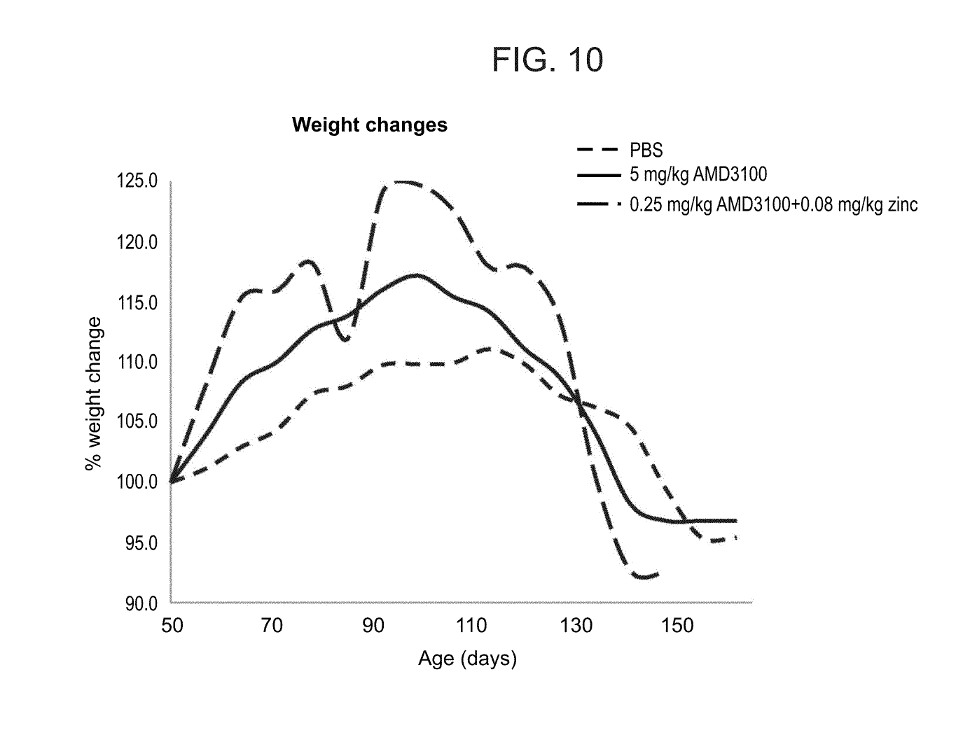

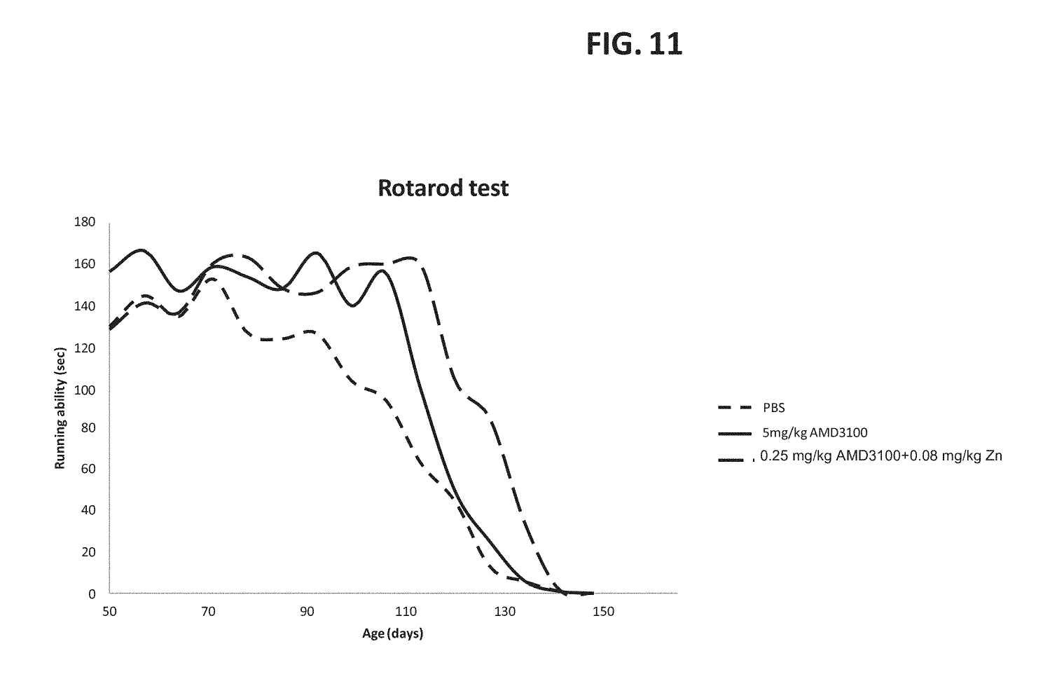

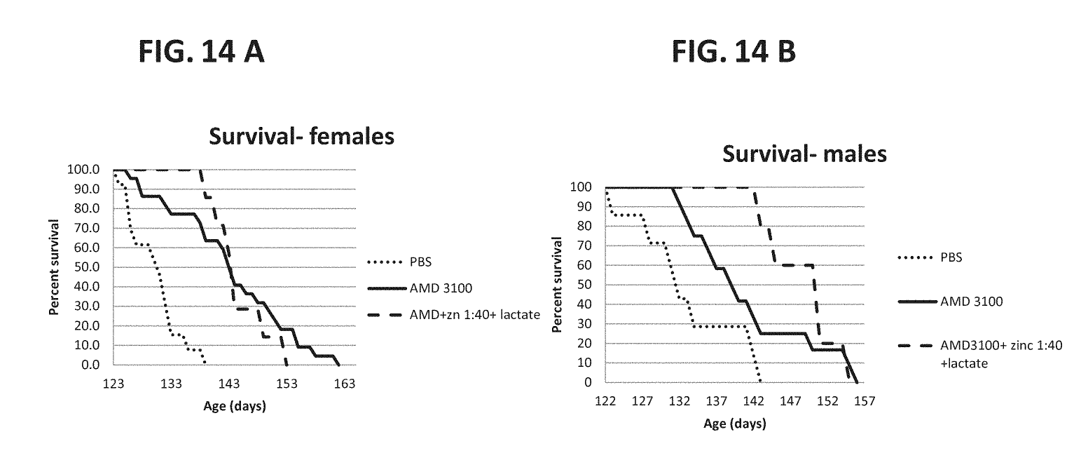

The present inventors have now shown that zinc and lactate both potentiate the therapeutic effect of AMD3100 on SOD1-G93A transgenic mice. More specifically, a complex of AMD3100 and Zinc was shown to significantly increase mouse body weight (FIG. 10) and improve mouse motor function (FIG. 11) and a mixture of AMD3100 and lactate was shown to delay disease onset cause a statistically significant lag in the time that the mice start to die (FIG. 1). The combination of AMD3100, Zinc and lactate was shown to have a synergistic effect (FIG. 14).

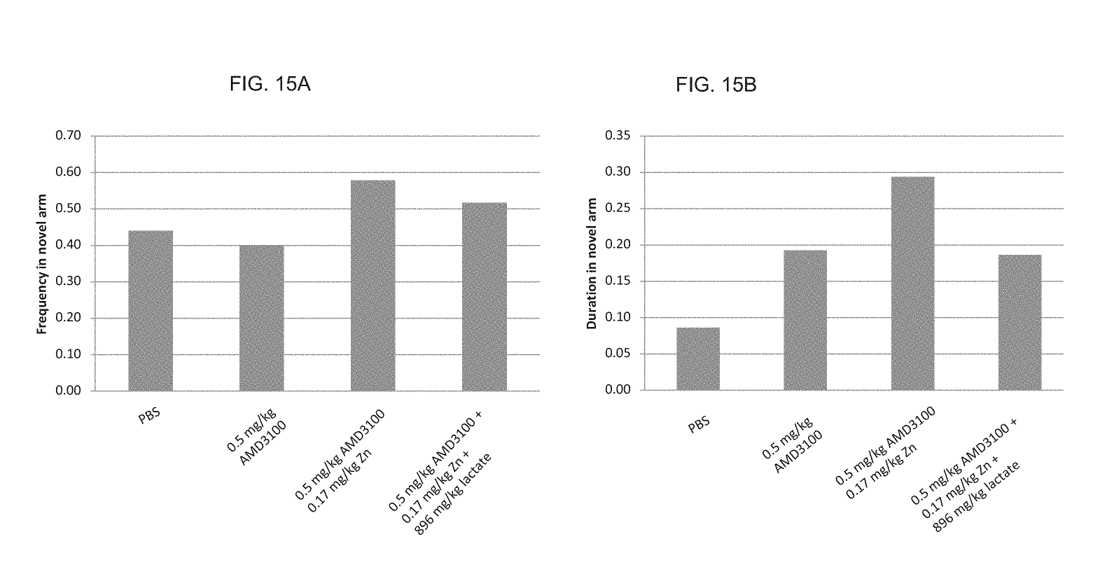

Whilst further reducing the present invention to practice, the present inventors showed that a complex of AMD3100 and Zinc also had beneficial effects for the treatment of another neurodegenerative disease—Alzheimers (FIGS. 15A-B).

Thus, according to a first aspect of the present invention, there is provided a method of treating a neurodegenerative disease in a subject in need thereof comprising administering to the subject a therapeutically effective amount of a CXCR4 antagonist and lactate, thereby treating the neurodegenerative disease.

As used herein, the term “neurodegenerative disease” refers to a condition characterized by a loss of neuronal function, structure, and/or neuron death. Examples of neurodegenerative diseases include, but are not limited to, Alexander disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease (Spinocerebellar ataxia type 3), Multiple sclerosis, Multiple System Atrophy, Neuroborreliosis, Parkinson disease, Pelizaeus-Merzbacher Disease, Pick's disease, Primary lateral sclerosis, Prion diseases, Refsum's disease, Sandhoff disease, Schilder's disease, Sub-Acute Combined Degeneration of the Cord Secondary to Pernicious Anaemia, Schizophrenia, Spielmeyer-Vogt-Sjogren-Batten disease (also known as Batten disease), Spinocerebellar ataxia (multiple types with varying characteristics), Spinal muscular atrophy, Steele-Richardson-Olszewski disease and Tabes dorsalis.

According to a particular embodiment, the neurodegenerative disease is a motor neuron disease.

The term “motor neuron disease” or “motoneuron disease” comprises a group of severe disorders of the nervous system characterized by progressive degeneration of motor neurons (neurons are the basic nerve cells that combine to form nerves). Motor neurons control the behavior of muscles. Motor neuron diseases may affect the upper motor neurons, nerves that lead from the brain to the medulla (a part of the brain stem) or to the spinal cord, or the lower motor neurons, nerves that lead from the spinal cord to the muscles of the body, or both. Spasms and exaggerated reflexes indicate damage to the upper motor neurons. A progressive wasting (atrophy) and weakness of muscles that have lost their nerve supply indicate damage to the lower motor neurons. Examples of motor neuron diseases include, but are not limited to, Progressive Bulbar Palsy, Amyotrophic Lateral Sclerosis (ALS), Spinal Muscular Atrophy, Kugelberg-Welander Syndrome, Lou Gehrig's Disease, Duchenne's Paralysis, Werdnig-Hoffmann Disease, Juvenile Spinal Muscular Atrophy, Benign Focal Amyotrophy and Infantile Spinal Muscular Atrophy.

As used herein, the term “CXCR4 antagonist” refers to an agent that is capable of blocking the binding of stromal cell-derived factor-1 (SDF1) to CXCR4.

In one embodiment, the CXCR4 antagonist is an anti-CXCR4 antibody.

In one embodiment, the CXCR4 antagonist is a CXCL12 analogue. CTCE-9908 and CTCE-0214 are peptide analogs of CXCL12 with inhibitory and agonist activity, respectively.

In another embodiment, the CXCR4 antagonist is a peptide. Exemplary peptide antagonists include LY2510924 (Galsky et al., Clin Cancer Res Jul. 1, 2014 20; 3581) T22, T134, T140, TN14003 and TC14012 (as disclosed by Burger et al., Leukemia (2009) 23, 43-52). Other peptide antagonists are disclosed by Portella et al., PLoS One. 2013; 8(9): e74548.

In another embodiment, the CXCR4 antagonist is a non-peptide antagonist such as the bicyclam AMD3100.

In further preferred embodiments, the CXCR4 antagonist is BMS-936564/MDX-1338, LY2510924, 1,1′-[1,4-phenylenebis(methylene)]bis [1,4,8,11-tetraazacyclotetradecane] (AMD3100; Plerixafor), N,N-dipropyl-N-[4-([[(1H-imidazol-2-yl)methyl)benzyl][(1-methyl-1H-imidazol-2-yl) methyl]amino]methyl)benzyl]-N-methylbutane-1, 4-diamine tri(2R,3R)-tartrate (KRH-3955), ([5-(4-methyl-1-piperazinyl)-2-({methyl[(8»S) -5,6,7,8-tetrahydro-8-quinolinyl]amino}methyl)imidazo[1,2-a]pyridin-3-yl]methanol) (GSK812397), or N-(1H-benzimidazol-2-ylmethyl)-N′-(5,6,7,8-tetrahydroquinolin-8-yl)butane-1,4-diamine (AMD11070).

The lactate may be a lactate salt or lactic acid.

The term “lactic acid” refers to the acid form of lactate, i.e., 2-hydroxypropionic acid.

The salt or dissociated form of lactate is specifically referred to herein as a “lactate salt,” for example, as the sodium (or calcium) salt of lactic acid or sodium lactate (or calcium lactate).

In one embodiment, the CXCR4 antagonist is administered prior to the lactate.

In another embodiment, the CXCR4 antagonist is administered following administration of the lactate. In still another embodiment, the CXCR4 antagonist is administered concomitantly with the lactate.

The CXCR4 antagonists of the present invention and the lactate are typically provided in combined amounts to achieve therapeutic and/or prophylactic effectiveness. This amount will evidently depend upon the particular compound selected for use, the nature and number of the other treatment modality, the condition(s) to be treated or prevented, the species, age, sex, weight, health and prognosis of the subject, the mode of administration, effectiveness of targeting, residence time, mode of clearance, type and severity of side effects of the CXCR4 antagonist and upon many other factors which will be evident to those of skill in the art.

In one embodiment, the lactate is typically used at a level between 10% of its normal minimum therapeutic dose and 200% of its maximum normal therapeutic dose. More preferably this range will be 25% of its normal minimum dose to 90% of its normal maximum dose.

In one preferred embodiment, the amount of the CXCR4 antagonist is below the minimum dose required for therapeutic or prophylactic effectiveness when used as a single therapy (e.g. 10-99%, preferably 25 to 75% of that minimum dose). This allows for reduction of the side effects caused by the CXCR4 antagonist but the therapy is rendered effective because in combination with the lactate, the combinations are effective overall.

Thus, for example if the CXCR4 antagonist is AMD3100, the dose is preferably less than 500 mg/kg, for example between 0.1-200 mg/kg or between 0.1-200 mg/kg, or between 10-150 mg/kg, or between 20-100 mg/kg.

The lactate is typically provided as an infusion solution (e.g. 500 ml or 1000 ml) of a 0.1-10 mmol/ml solution of lactate, more preferably of 0.1-1 mmol/ml solution for example about 0.5 mmol/ml or 0.6 mmol/ml. An exemplary dose of lactate is between 100-10,000 mg/kg, more preferably between 100-2000 mg/kg, 500-1000 mg/kg.

In one preferred aspect of the present invention, the CXCR4 antagonist and the lactate are synergistic with respect to their dosages. That is to say that the effect provided by the CXCR4 antagonist is greater than would be anticipated from the additive effects of the CXCR4 antagonist and the lactate when used separately. In an alternative but equally preferred embodiment, the CXCR4 antagonist and the lactate are synergistic with respect to their side effects. That is to say that the side-effects caused by the CXCR4 antagonist in combination with the lactate are less than would be anticipated when the equivalent therapeutic effect is provided by either the CXCR4 antagonist or by the lactate when used separately.

The CXCR4 antagonist, together with the lactate may be administered with additional agents to enhance their therapeutic effect. In one embodiment, the additional agent is zinc. The zinc may be administered as a separate entity, or in the case where the CXCR4 antagonist is AMD3100, a zinc chelator, the zinc may be complexed with the AMD3100.

According to another aspect of the present invention there is provided a method of treating a neurodegenerative disease in a subject in need thereof comprising administering to the subject a therapeutically effective amount of AMD3100 and zinc, thereby treating the neurodegenerative disease.

In one embodiment, the zinc of this aspect of the present invention is provided as a salt.

According to one embodiment, the AMD3100 is administered prior to the zinc. In another embodiment, the AMD3100 is administered following administration of the Zinc.

In still another embodiment, the AMD3100 is administered concomitantly with the lactate. In this embodiment, the AMD3100 may be administered as a complex with the zinc.

To prepare a complex of AMD3100 and zinc, typically the two may be combined at a molar ratio of about 1:1-1:10, more preferably between 1:1-1:5, for example at a ratio of about 1:2. Since AMD3100 is a natural chelator of zinc, a complex will be generated.

AMD3100 and the zinc are typically provided in combined amounts to achieve therapeutic and/or prophylactic effectiveness. This amount will evidently depend upon the particular compound selected for use, the nature and number of the other treatment modality, the condition(s) to be treated or prevented, the species, age, sex, weight, health and prognosis of the subject, the mode of administration, effectiveness of targeting, residence time, mode of clearance, type and severity of side effects of AMD3100 and upon many other factors which will be evident to those of skill in the art.

The zinc is typically be used at a level between 10% of its normal minimum therapeutic dose and 200% of its maximum normal therapeutic dose. More preferably this range will be 25% of the normal minimum dose to 90% of the normal maximum dose.

The amount of zinc is typically between 0.1-100 μg/kg and more preferably between about 0.1-10 μg/kg.

In one preferred embodiment, the amount of AMD3100 is below the minimum dose required for therapeutic or prophylactic effectiveness when used as a single therapy (e.g. 10-99%, preferably 25 to 75% of that minimum dose). This allows for reduction of the side effects caused by AMD3100 but the therapy is rendered effective because in combination with the zinc, the combinations are effective overall.

Thus, for example the dose of AMD3100 is preferably less than 500 μg/kg, more preferably less than 240 μg/kg, for example between 0.1-200 μg/kg or between 10-150 μg/kg. The CXCR4 antagonist (e.g. AMD3100), lactate, and/or zinc may be administered to an organism per se, as a single pharmaceutical composition, or as individual pharmaceutical compositions where they are mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism. Herein the term “active ingredient” refers to the CXCR4 antagonist/zinc/lactate accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

According to a particular embodiment, when the CXCR4 antagonist is AMD3100, the route of administration is subcutaneous.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

According to a particular embodiment, at least one of the agents is administered into the muscle of the subject.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran.

Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to ensure that muscle or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient.

Thus for example the CXCR4 antagonists (e.g. AMD3100) may be provided in kits together with either one or both of the zinc or lactate.

The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

It is expected that during the life of a patent maturing from this application many relevant CXCR4 antagonists will be developed and the scope of the term CXCR4 antagonists is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization-A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### AMD3100 and lactate for treatment of ALS

Experimental set-up: 50 day old female SOD1-G93A mice were treated with either 5 mg/kg AMD3100 alone, 896 mg/kg Na-L-lactate alone or a combination of 5 mg/kg mM AMD3100 and 896 mg/kg Na-L-lactate twice a week, starting at 50 days old (for ALS), until death. All treatments were administered subcutaneously. The mice were weighed once a week following the various treatments and tested for motor function, also once a week. The time taken until death was recorded.

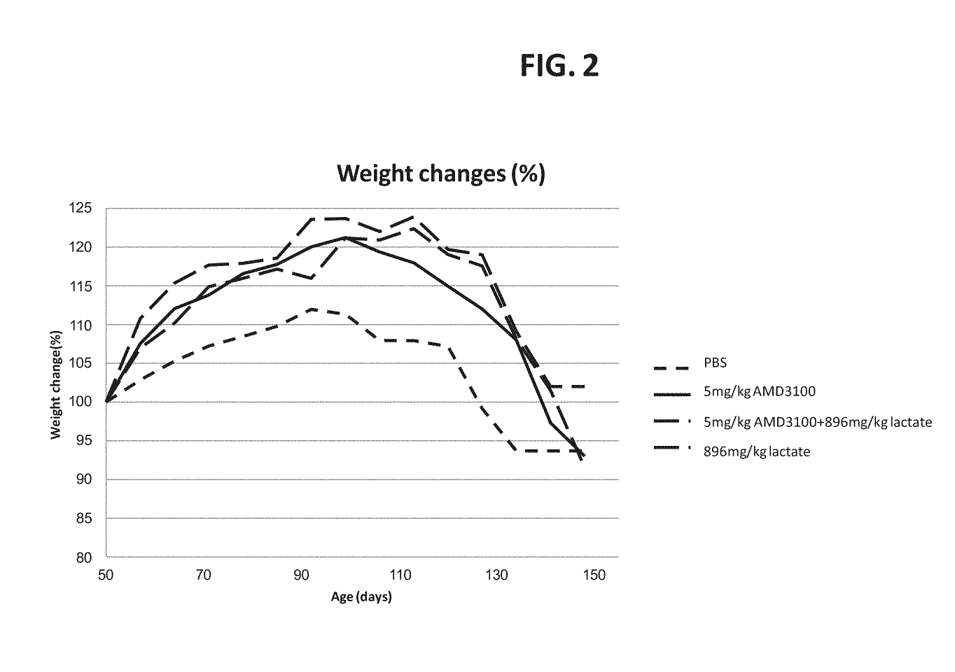

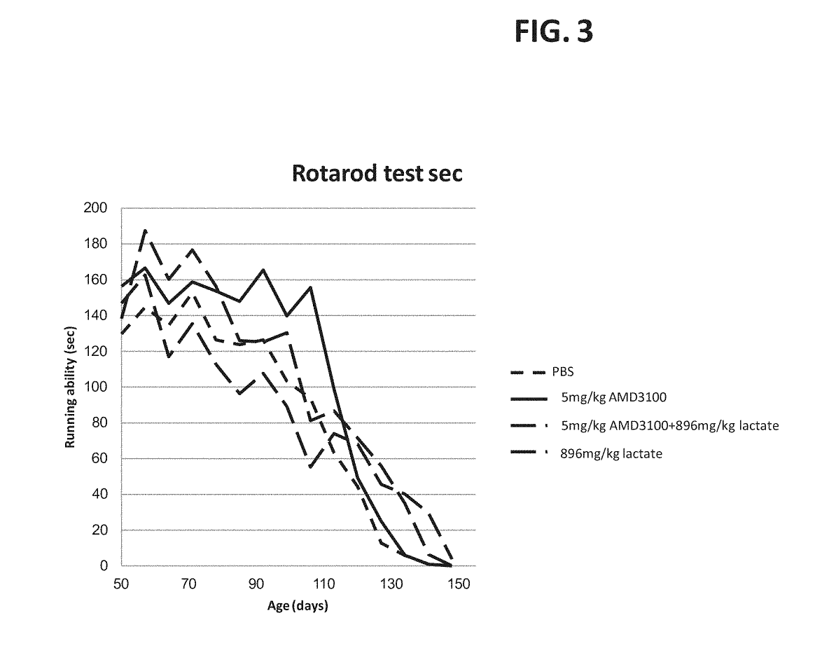

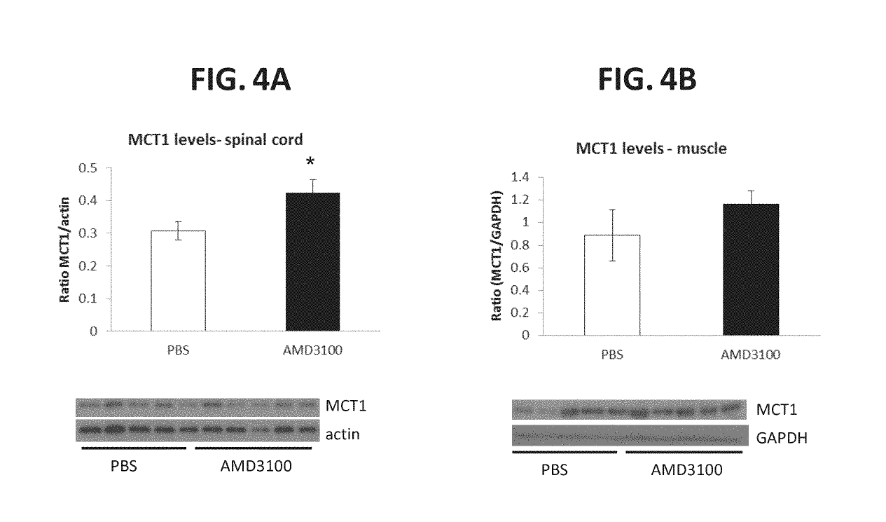

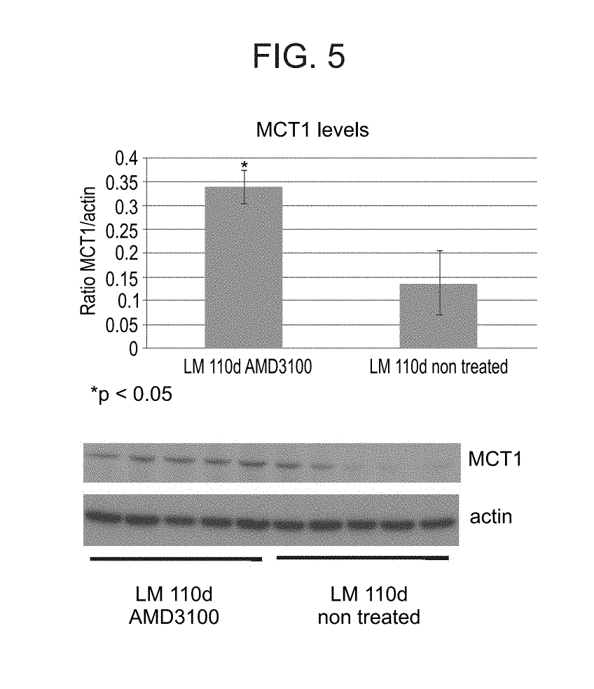

Results: The combined treatment showed a synergistic effect by increasing the survival, weight and motor functions, in addition to causing a lag in time until death. 100% of mice responded to the treatment (FIGS. 1-3). Furthermore, treatment of 5 mg/kg AMD3100 alone in SOD1-G93A mice and littermates thereof showed an increase in MCT1 levels, providing an explanation for the synergistic effect of lactate and AMD3100 (FIGS. 4 and 5).

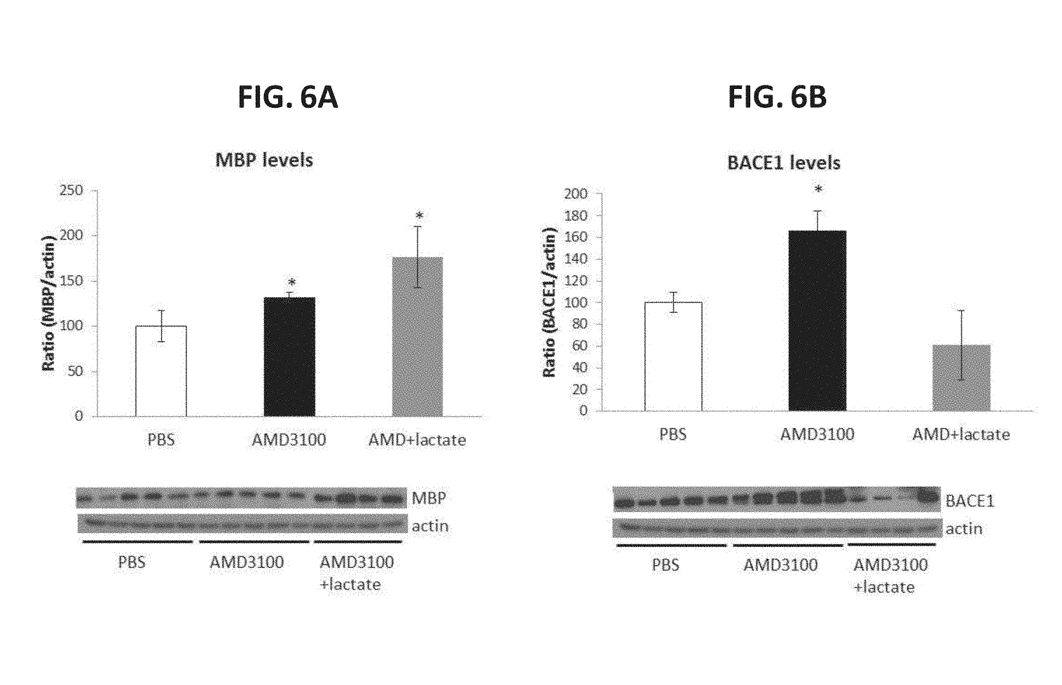

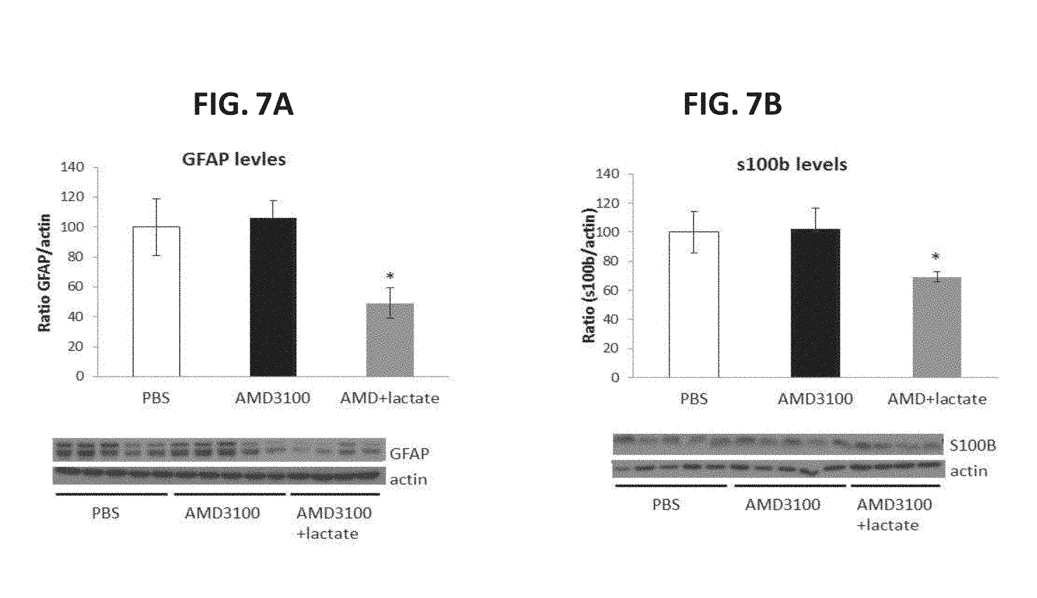

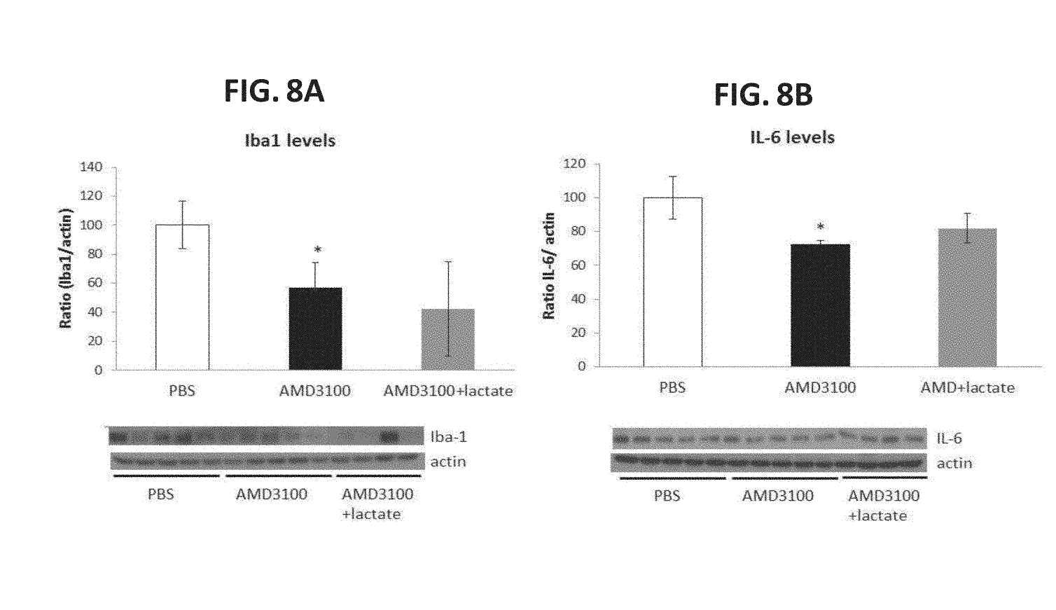

FIGS. 6A-B illustrate the change in MBP and BACE1, respectively, following 5 mg/kg AMD3100 treatment versus 5 mg/kg AMD3100+896 mg/kg lactate treatment in 50 day old female mice of SOD1-G93A mice. FIGS. 7A-B illustrate the change in activation of astrocytes following 5 mg/kg AMD3100 treatment versus 5 mg/kg AMD3100+896 mg/kg lactate treatment in 50 day old female mice of SOD1-G93A mice. Specifically, FIG. 7A shows GFAP levels and FIG. 7B shows S100B levels. FIGS. 8A-B illustrate the change in microglial reactivity following 5 mg/kg AMD3100 versus 5 mg/kg AMD3100+896 mg/kg lactate treatment in 50 day old female mice of SOD1-G93A mice. Specifically, FIG. 8A shows Iba-1 levels whilst FIG. 8B shows IL-6 levels.

### Example 2

### AMD3100 and zinc for treatment of ALS

Experimental set-up: 50 day old female SOD1-G93A mice were treated with either 5 mg/kg AMD3100 alone or a complex of 0.25 mg/kg AMD3100+0.08 mg/kg Zinc or 0.125 mg/kg AMD3100+0.04 mg/kg Zinc twice a week, starting at 50 days old until death. All treatments were administered subcutaneously. The mice were weighed once a week following the various treatments and tested for motor function, also once a week. The time taken until death was recorded.

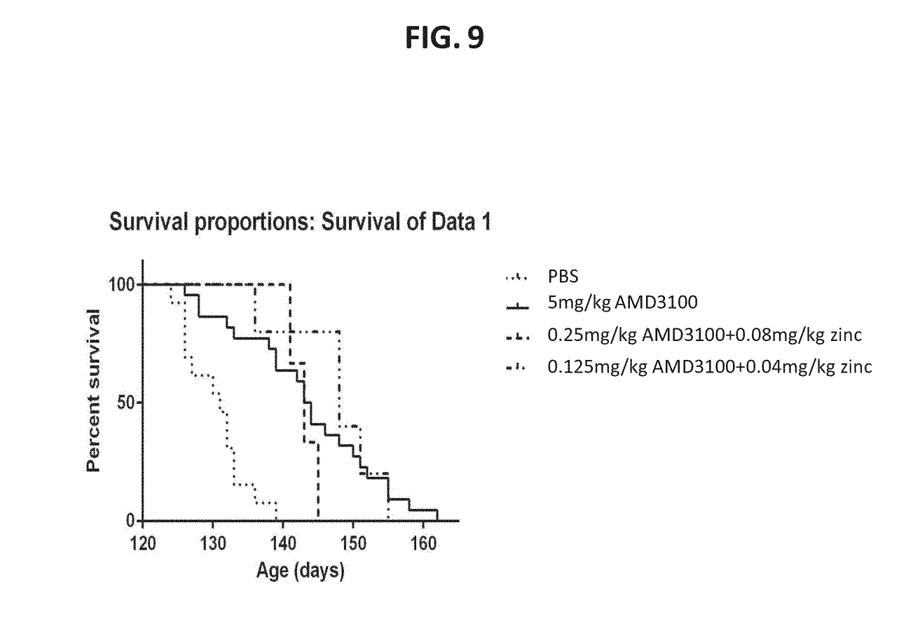

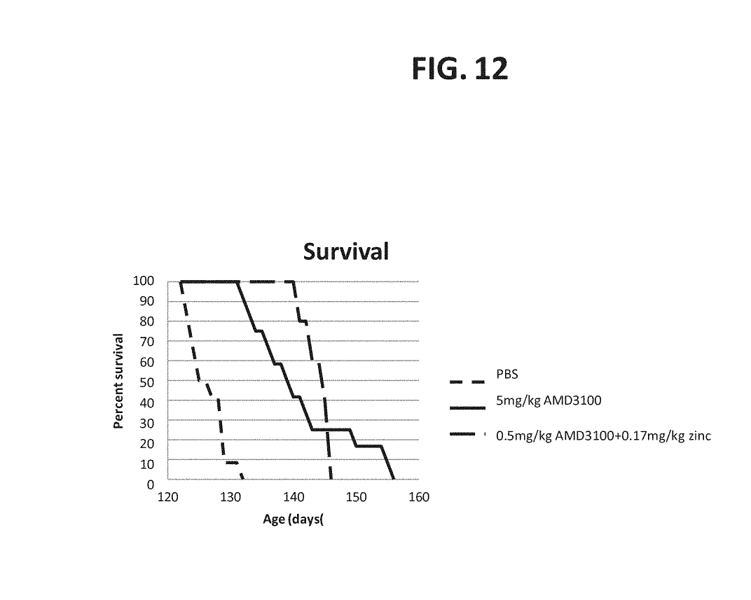

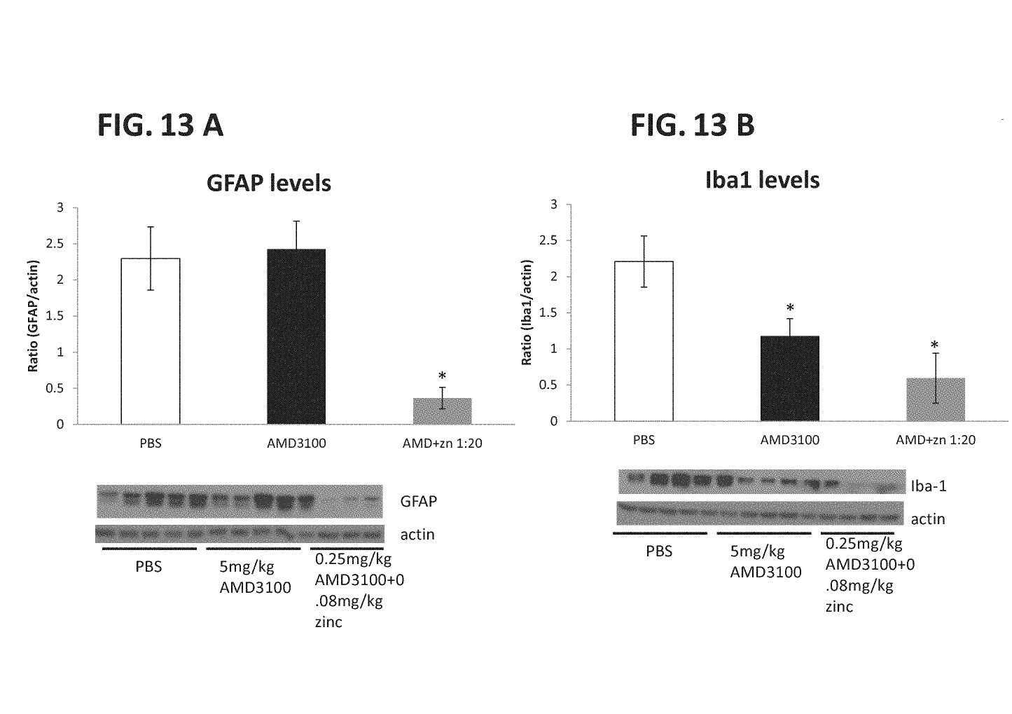

Results: The complex showed a beneficial effect by increasing the survival, weight and especially motor functions (FIGS. 9-12). FIGS. 13A-B illustrate the change in inflammation following 5 mg/kg AMD3100 versus 0.25 mg/kg AMD3100+0.08 mg/kg zinc treatment in 50 day old female mice of SOD1-G93A mice. Specifically, FIG. 13A illustrates GFAP levels, whilst FIG. 13B illustrates Iba-1 levels.

### Example 3

### AMD3100-zinc and lactate for treatment of ALS

Experimental set-up: 50 day old male SOD1-G93A mice were treated with either 5 mg/kg AMD3100 alone or a complex of 0.5 mg/kg AMD3100+0.17 mg/kg Zinc+896 mg/kg lactate twice a week, starting at 50 days old, till death. All treatments were administered subcutaneously. The time taken until death was recorded.

Results: The complex showed a beneficial effect by increasing the survival by 19 days compared to PBS, and by 5 days compared to 5 mg/kg AMD3100 alone (FIG. 14).

### Example 4

### AMD3100-zinc (and lactate) for treatment of Alzheimer's Disease

Experimental set-up: 9 month old 3×Tg-AD mice were treated with PBS or 0.5 mg/kg AMD3100 or 0.5 mg/kg AMD3100 0.17 mg/kg Zn or 0.5 mg/kg AMD3100+0.17 mg/kg Zn+896 mg/kg lactate once a week. All treatments were administered subcutaneously. After 3 month of treatment, all mice were subjected to Y maze test in order to evaluate their cognitive function. Each mouse was allowed to explore two arms of the Y maze for 5 min, rested for another 5 min, and then explored all 3 arms of the Y maze for another 5 min.

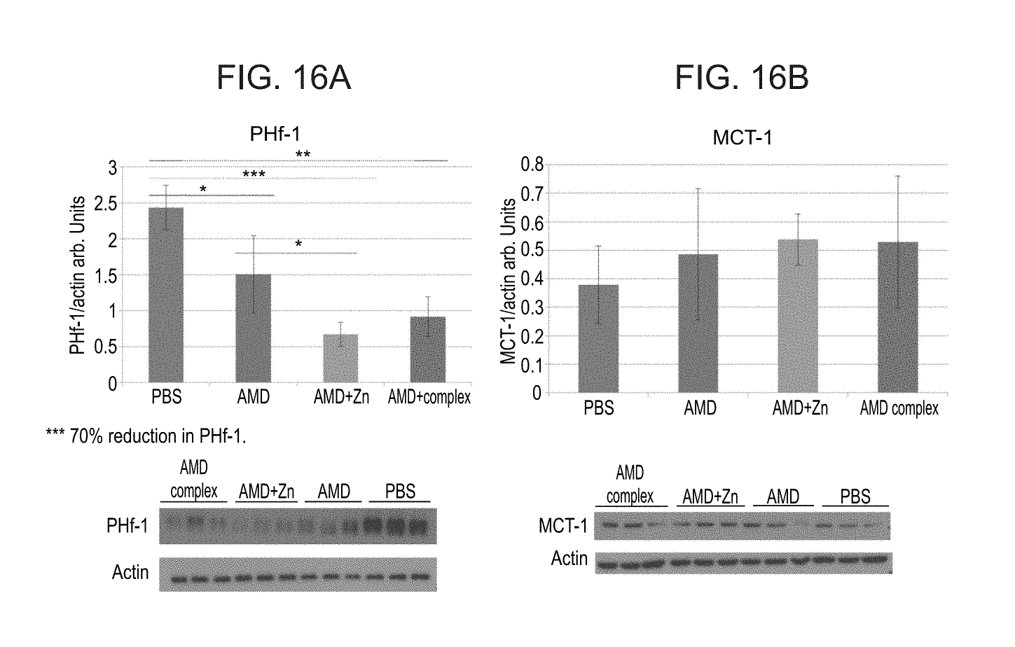

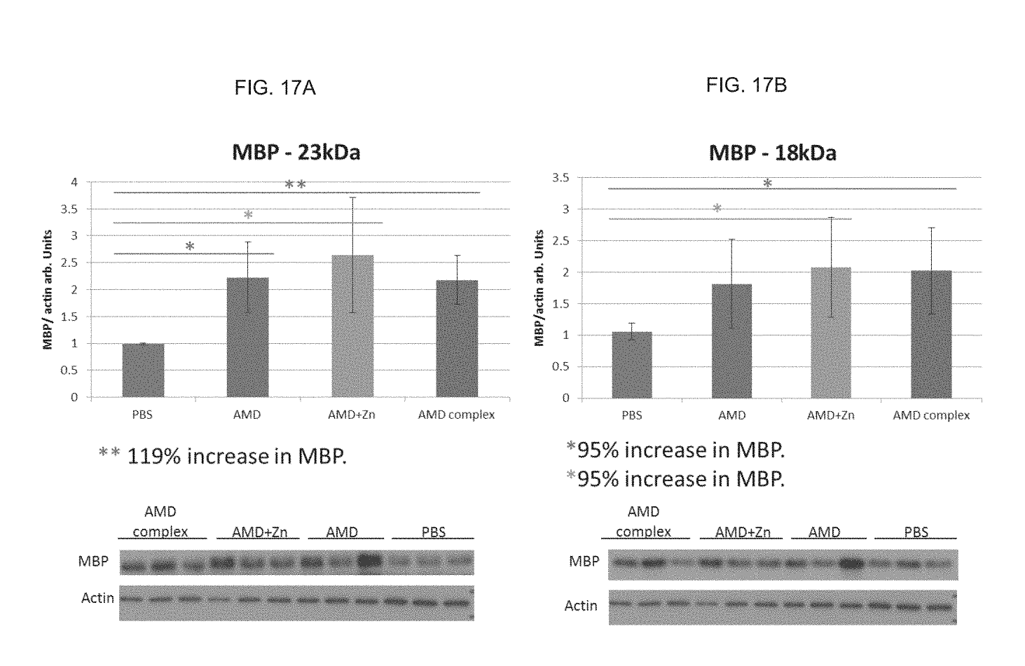

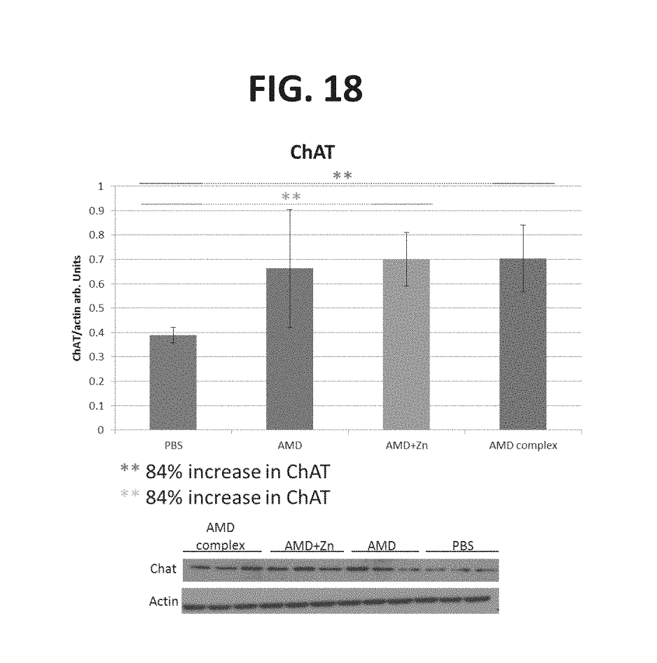

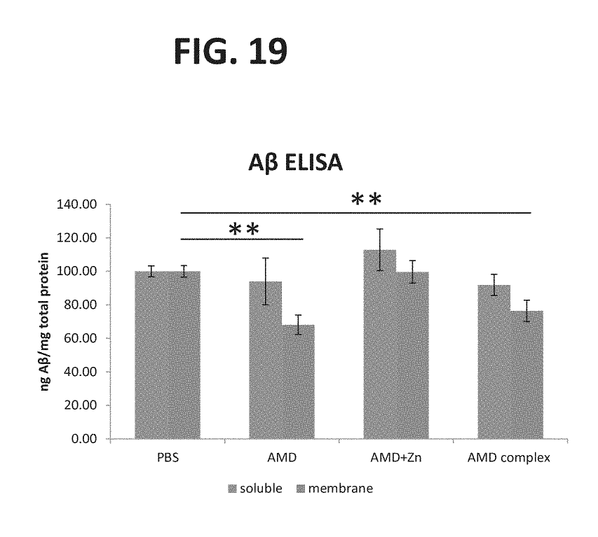

Results: Female 3×Tg-Ad mice treated with 0.5 mg/kg AMD3100 0.17 mg/kg Zn or 0.5 mg/kg AMD3100+0.17 mg/kg Zn+896 mg/kg lactate showed improvement in their cognitive function (FIGS. 15A-B). FIGS. 16A-B are bar graphs illustrating the changes in PHF-1 and MCT-1 results of 3×Tg-AD mice. Specifically, FIG. 16A illustrates that the reduction in PHF-1 is pronounced in AMD+Zn and AMD+complex compared to AMD only. FIG. 16B shows that a trend for increase in MCT-1 was observed, although it was not statistically significant. FIGS. 17A-B are bar graphs illustrating the changes in myelin binding protein (MBP) isotypes. Specifically, FIG. 17A illustrates the increase in MBP-23 kDa isotype, whilst FIG. 17B illustrates the increase in MBP-18 kDa isotype. FIG. 18 are bar graphs illustrating the changes in ChAT. Increase in ChAT was statistically significant in AMD+Zn and AMD+complex treatment groups only. FIG. 19 is bar graphs illustrating the changes in AP by ELISA.

Reduction in AP was statistically significant in AMD and AMD+complex treatment groups in the membrane fraction.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating a neurodegenerative disease in a subject in need thereof comprising administering to the subject a therapeutically effective amount of a CXCR4 antagonist and lactate, thereby treating the neurodegenerative disease.

2. The method of claim 1, wherein said neurodegenerative disease is selected from the group consisting of amyotrophic lateral sclerosis (ALS), Parkinson's disease (PD), Alzheimer's disease (AD), Huntington's disease, multiple sclerosis (MS), Creutzfeldt-Jacob disease (CJD), epilepsy, stroke, autoimmune encephalomyelitis, diabetic neuropathy and glaucomatous neuropathy.

3. The method of claim 1, wherein said neurodegenerative disease is ALS.

4. The method of claim 1, wherein said CXCR4 antagonist is selected from the group consisting of AMD3100 (plerixafor) BKT140, TN14003, CTCE-9908, KRH-2731, TC14012, KRH-3955, and AMD070.

5. The method of claim 1, wherein said CXCR4 antagonist is AMD3100.

6. The method of claim 1, wherein said lactate is administered concomitantly with said CXCR4 antagonist.

7. The method of claim 1, wherein said lactate is administered prior to or following said CXCR4 antagonist.

8. The method of claim 5, wherein the dose of said AMD3100 is less than 240 μg/kg.

9. The method of claim 5, wherein the dose of said AMD3100 is between 0.1-500 μg/kg.

10. The method of claim 5, wherein the dose of said AMD3100 is between 10-150 μg/kg.

11. The method of claim 1, wherein said CXCR4 antagonist is administered subcutaneously.

12. The method of claim 1, further comprising administering to the subject zinc.

13. The method of claim 5, wherein said AMD3100 is complexed with zinc.

14. A method of treating a neurodegenerative disease in a subject in need thereof comprising administering to the subject a therapeutically effective amount of AMD3100 and zinc, with the proviso that the neurodegenerative disease is not ALS, thereby treating the neurodegenerative disease.

15. The method of claim 14, wherein said neurodegenerative disease is selected from the group consisting of Parkinson's disease (PD), Alzheimer's disease (AD), Huntington's disease, multiple sclerosis (MS), Creutzfeldt-Jacob disease (CJD), epilepsy, stroke, autoimmune encephalomyelitis, diabetic neuropathy and glaucomatous neuropathy.

16. (canceled)

17. The method of claim 14, wherein said zinc is administered concomitantly with said AMD3100.

18. The method of claim 17, wherein said zinc is complexed with said AMD3100 prior to said administering.

19. The method of claim 14, wherein said zinc is administered prior to, or following said AMD3100.

20. The method of claim 14, further comprising administering to the subject lactate.

21. The method of claim 14, wherein the dose of said AMD3100 is less than 240 μg/kg.

22. The method of claim 14, wherein the dose of said AMD3100 is between 0.1-500 μg/kg.

23. The method of claim 14, wherein the dose of said AMD3100 is between 10-150 μg/kg.

24. The method of claim 14, wherein said AMD3100 is administered subcutaneously.

25-32. (canceled)
